(19) United States
(12) Patent Application Publication
(10) Pub. No.: US 2006/0276840 A1
(43) Pub. Date: Dec. 7, 2006

(54) Methods and devices for treating dural puncture

(76) Inventors: Yakov Perper, Valley Stream, NY (US); Grigoriy Kreymerman, Pembroke Pines, FL (US)

Correspondence Address: MARTIN NOVACK, 16355 VINTAGE OAKS LANE, DELRAY BEACH, FL, 33484, US

(21) Appl. No.: 11/430,257

(22) Filed: May 8, 2006

Related U.S. Application Data
(60) Provisional application No. 60/679,268, filed on May 9, 2005.

(51) Int. Cl. A61B 17/08

(52) U.S. Cl. 606/215; 424/447

(57) ABSTRACT

A device for repairing a dural puncture, including: a patch formed of collagen type of material that includes a bulbous head portion that is larger than the puncture opening, and a neck portion that is proportioned to fit through the puncture opening.

### PRIORITY CLAIM

The present Application claims priority from U.S. Provisional Patent Application No. 60/679,268, filed May 9, 2005, and said U.S. Provisional Patent Application is incorporated herein reference.

### FIELD OF THE INVENTION

This invention relates to prevention of Postdural Puncture Headache (PDPH) after inadvertent or elective dural puncture, and to devices and methods for repairing dural puncture.

### BACKGROUND OF THE INVENTION

Postdural puncture headache (PDPH) is an iatrogenic complication of neuraxial anesthesia and results from the puncture of the dura mater. The signs and symptoms of PDPH result from loss of cerebrospinal fluid, traction on the cranial contents, and reflex cerebral vasodilatation. As female sex and young age are purported risk factors, the complication is common in the obstetrical population, which frequently receives epidural or spinal analgesia and anesthesia during labor and delivery.

PDPH can be, and often is, a very distressing and debilitating symptom. PDHP is typically exacerbated by assuming the upright position and relieved by recumbence. The site of headache is not diagnostic, as it can be occipital, frontal, unilateral or on top of the head. Other associated symptoms include nausea, vomiting, dizziness and photophobia. It can typically present itself from between a few hours to two days after dural puncture. Presentation later than this is unusual and tends to result in a less severe headache, which resolves relatively soon. Without treatment, PDPH can persist for several days and, on rare occasions, for weeks or even months. The group of patients most frequently focused on is those in obstetrics. Because the symptoms are debilitating at a time when the new mother (who typically does not have another illness, unlike patients who need a diagnostic lumbar puncture) has no wish to be recumbent in bed, early diagnosis and treatment are imperative.

The causative mechanism of the PDPH is believed to be associated with the continuing leakage of cerebrospinal fluid (CSF) through the dural opening left by an epidural or spinal needle. The leakage of CSF causes a decrease in CSF pressure, which, in turn, produces compensatory cerebral vasodilatation. Bringing the patient into the erect position also results in traction on the pain-sensitive, dilated blood vessels. Accordingly, conservative therapy for the PDPH consists of bed rest and analgesics. The current treatments for PDPH are (a) conservative treatment: bed rest, analgesics, a lot of fluids, caffeine; (b) “blood patch”.

In an attempt to minimize leakage of CSF, an available procedure is to create what is known as a blood patch. This is done by obtaining 10 to 20 cc of blood from the patient and injecting this volume of blood into the tissue adjacent the puncture site of the epidural or spinal needle. This relatively large volume of blood is required since it is virtually impossible for the health care professional to exactly position the blood patch directly over the original puncture site. In effect, therefore, the blood patch is designed to seal the dural puncture thereby significantly minimizing the frequency of the postdural headache. The effectiveness of the blood patch is approximately 85-95%. If a first procedure is not successful, then the second blood patch procedure can be implemented. The effectiveness of the second blood patch is even higher: 90-99%.

The professional doing labor epidural can recognize inadvertent dural puncture by the flush of the fluid through the hub of the epidural needle. There is usually no great difficulty in following up those patients who are known to have had the dura breached (see the description of PDPH above). However, inadvertent dural puncture sometimes goes unrecognized. A clinical picture of PDPH typically develops on the second or third day after a performed or attempted epidural in such a case.

Spinal dura is a firm collagen tissue and is the continuation of the brain dura mater. It isolates the spinal column, nerves, vessels and cerebro-spinal fluid. The dura is firm enough to prevent CSF leakage, but can be easily penetrated by the epidural needle. The dura is surrounded by epidural space which is between the dura and spinal ligaments. The epidural space contains nerves, vessels, and loose adipose tissue and is characterized by low pressure (sub atmospheric or atmospheric); which is the reason that the most common way to identify the epidural space is the “loss of resistance” technique. The young, healthy parturient is a challenge for health care professional. This is due to the fact that her tissues are relatively soft and pliable, so the loss of resistance can go unrecognized (especially when the medical practitioner has limited experience). This is a reason that the number of inadvertent dural puncture can be significant (up to 6%) in some hospitals. This number is typically higher in the teaching institutions (those that have residency programs) and lower in private practices. Even a very experienced anesthesiologist can develop an inadvertent dural puncture (especially if the patient has difficult anatomy; morbid obesity for example), but in a much lower number of cases (below 1%). A substantial number of inadvertent dural punctures have gone unreported due to the nature of the complication.

Currently, when there is a recognized dural puncture (the practitioner sees CSF leakage from the hub of the epidural needle or the practitioner withdraws the CSF on suction from the distal end of the epidural catheter inadvertently placed into the intrathecal space), the health care professional may: (a) Withdraw the epidural needle and redo the epidural on another level. In this case the practitioner will discuss the complication with the patient and the treatment available: bed rest, fluids, analgesics and if conservative treatment doesn't help, then the practitioner may implement a “blood patch”. The risk of development of PDPH in this case is approximately 75-85%. (b) Put the catheter through the epidural needle into the intrathecal space and leave it there. The practitioner can use the intrathecal catheter later, for example for labor (continuous intrathecal labor anesthesia) or for the surgery (emergent or elective cesarean section) to decrease the possibility of development of PDPH. Leaving the epidural catheter for 12-20 hours in the intrathecal space could decrease the incidence of PDPH significantly (see K. M. Kuczkowski, MD Acta Anaesthisiologica Scand, 2005). The prevailing theory as to why this happens is that the catheter creates inflammation at the dural hole and, after it is removed, the closure of the dural hole is accelerated.

It is among the objects of the present invention to provide solutions to problems associated with dural puncture.

### SUMMARY OF THE INVENTION

In accordance with a form of the invention, a device is provided for repairing a dural puncture, including: a patch formed of collagen type of material that includes a bulbous head portion that is larger than the puncture opening, and a neck portion that is proportioned to fit through the puncture opening. In an embodiment of this form of the invention, the collagen type of material can comprise, for example, collagen, fibrin, or Duragen. The patch can, optionally, contain medication, such as a time release medication. Also, in an embodiment of this form of the invention, a suture tail is coupled with said neck portion.

In accordance with another form of the invention, a system is provided for sealing a dural puncture, including: a hollow needle; an elongated catheter within the needle, the catheter having a distal end within the front tip of the needle and a proximal end extending from the rear of the needle; and a collagen-type material at the distal end of the catheter; whereby when the needle is inserted through a puncture in the dura mater, the proximal end of the catheter can be manipulated to urge the collagen-type material out of the needle tip to form a seal of the puncture. In an embodiment of this form of the invention, the collagen-type material comprises a coating of collagen-type material on the distal end of the catheter. In another embodiment of this form of the invention, the collagen-type material comprises a patch formed of collagen type of material that includes a bulbous head portion and a neck portion that is proportioned to fit through the puncture opening. In this embodiment, the patch is folded to fit within the needle, and the neck portion of the patch is within the distal end of the catheter.

In accordance with a form of the method of the invention, a technique is set forth for sealing a dural puncture, including the following steps: providing a needle and an elongated catheter within the needle, the catheter having a distal end within the front tip of the needle and a proximal end extending from the rear of the needle; providing a collagen-type material at the distal end of the catheter; inserting the needle through a puncture in the dura mater; and manipulating the proximal end of the catheter to urge the collagen-type material out of the needle tip to form a seal of the puncture. In an embodiment of this form of the method of the invention, the step of providing a collagen-type material at the distal end of the catheter comprises coating the distal end of the catheter with a collagen-type material, said coating of collagen-type material being adapted to be larger than said puncture when it becomes soaked with spinal fluid. In another embodiment of this form of the method of the invention, the step of providing a collagen-type material at the distal end of the catheter comprises providing a patch formed of collagen type of material that includes a bulbous head portion and a neck portion that is proportioned to fit through the puncture opening, the patch being folded to fit within the needle.

Further features and advantages of the invention will become more readily apparent from the following detailed description when taken in conjunction with the accompanying drawings.

### BRIEF DESCRIPTION OF THE DRAWINGS

FIG. 1 shows a collagen dural patch in the unfolded position.

FIG. 2A is a diagram, in cross section, showing operation of an embodiment of the invention in conjunction with a spinal needle inserted between spinal ligaments to puncture the dural wall.

FIG. 2B shows, in further detail, the embodiment of FIG. 2A.

FIGS. 3A and 3B illustrate operation in accordance with an embodiment of the invention, whereby a folded collagen patch is inserted to plug a puncture in the dural wall.

FIGS. 4A, 4B, and 4C illustrate operation in accordance with another embodiment of the invention, wherein a collagen coated catheter is used plug a puncture in the dural wall.

### DETAILED DESCRIPTION

Two devices are disclosed in accordance with embodiments of the invention, and which can be used in practicing methods in accordance with the invention. Both devices can be used for prevention of PDPH: (1) Device with collagen dural patch; (2) Device with collagen coated catheter. These devices could be part of every epidural kit.

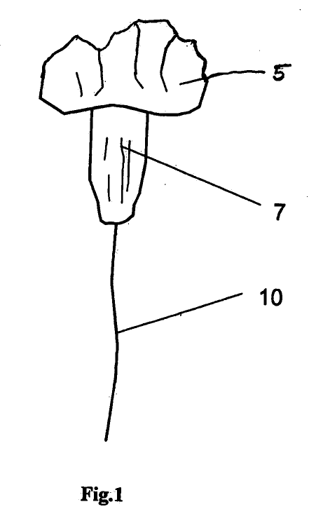

A collagen dural patch in unfolded position is shown in FIG. 1. The collagen dural patch has head 5, neck 7 and biodegradable suture (tail) 10 attached to the neck. This patch is well suited to prevent the development of Post Dural Puncture Headache in labor and delivery. However this invention can be used in any other area of medicine or surgery where epidural or spinal procedures are employed.

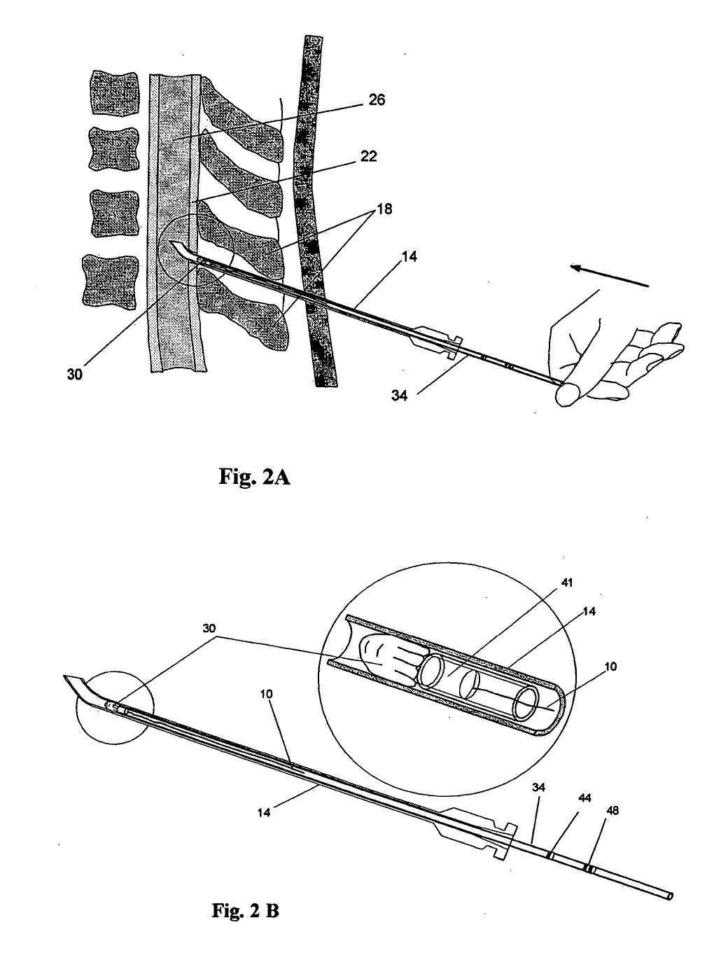

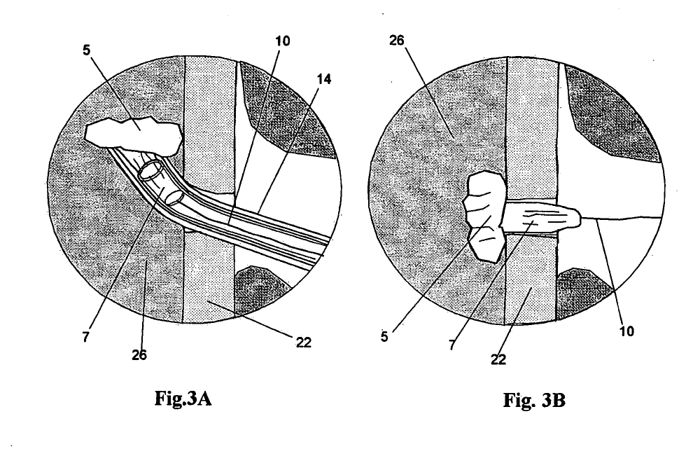

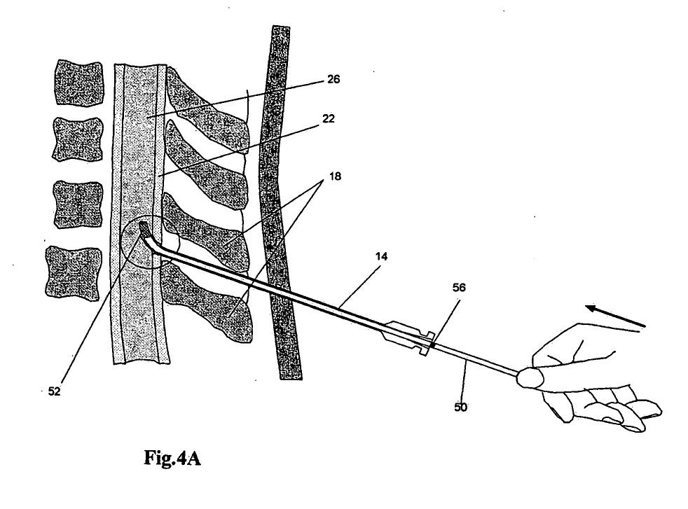

A device with the collagen dural patch works as described below. During anesthesia (FIG. 2A) the epidural (or spinal) needle 14 inserted between spinal ligaments 18 unintentionally (or intentionally) can puncture the dural wall 22 into the intrathecal space 26. In this embodiment, the collagen patch 30 is placed inside the epidural needle, on the distal end of the catheter 34, in a folded position, and prevents leakage of the cerebrospinal fluid from the intrathecal space (FIG. 2B). The neck (7) of the folded collagen patch is inserted into the distal end 41 of the catheter. The catheter has two marks on its proximal end. The first mark 44 corresponds to the length of the epidural needle and the second mark 48 indicates when the head 5 of the collagen dural patch exits the tip of the epidural needle to unfold (FIG. 3A). The epidural needle will withdraw when the inserted catheter reaches the second mark 48 and the collagen patch is unfolded. Then, the catheter will be removed slowly. The neck 7 of the collagen dural patch inside the tip of the catheter will be introduced to and plug the puncture in the dural wall during withdrawal of the catheter. The neck 7 will release from the catheter and plug the hole as the unfolded head 5 of the collagen patch anchors on the inner wall of the dura (FIG. 3B). The biodegradable suture (tail) 10 extends from the neck of the dural patch (for example:1-inch vicryl). The suture 10, anchored in the ligamentum flavum, helps retain the position of the collagen dural patch until the puncture in the dura heals.

The collagen dural patch of this embodiment has the following properties: (1) It provides effective protection against cerebrospinal fluid (CSF) leakage when in place; (2) It is substantially inert to the human body; in other words it doesn't tend to trigger an immune response; (3) It is fully resorbed following complete tissue closure of the dural defect; (4) It causes minimal adhesion formation when resolved; and (5) It is a 3D matrix, which promotes cell growth with rapid expansion of cell population.

The material “Duragen” is a collagen patching material that is used in spinal surgery and is an example of a material that can be used to form the “dural patch” hereof.

A further embodiment hereof comprises a collagen-coated catheter. The collagen-coated catheter CCC or fibrin-coated catheter FCC is a catheter that has the proximal end covered with collagen or fibrin respectively. Both collagen and fibrin are used widely in medicine: for example duragen is used in spinal surgery to patch externally the dural hole after dural damage during the spinal surgery and fibrinogen patches are used to stop bleeding from soft tissues where hemostasis cannot be otherwise achieved (liver or spine, for example). Based on the information available, our preference is the collagen-coated catheter. However, clinical testing can demonstrate which one is superior and safer for the prevention of PDPH.

The CCC embodiment can be used, for example, in the following circumstances: (a) When there is the need for continuous intraspinal anesthesia/analgesia (continuous labor analgesia or spinal anesthesia for revascularization of the lower extremities or long-term intraspinal analgesia in cancer patients). (b) To treat postoperative pain in patients in whom spinal analgesia can be advantageous compared to the standard practice (PCA, epidural) or when the common remedies cannot control the pain. (c) When, accidentally, the epidural needle was placed in the intrathecal space and there is a possibility of surgery (in case of labor and delivery there is always a risk of the emergency caesarian section). The CCC catheter can provide quick spinal anesthesia and can prevent the development of PDPH. (d) When the health care practitioner decided to leave the catheter on purpose after accidental dural puncture: to decrease the risk of the PDPH (see further below).

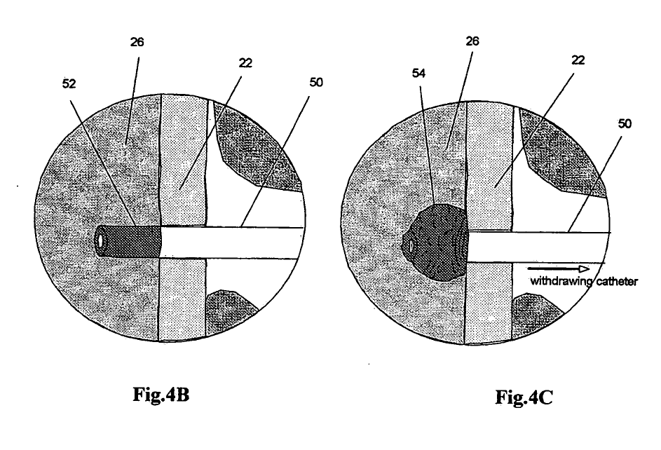

CCC can decrease the risk of the development of PDPH in the following ways: (1) By the physical presence of the catheter in the dural hole. This is due to decreasing the room for CSF leak. (2) The catheter promotes cell growth at the dural hole and that speeds up the closure of the dural hole (possibly by inducing inflammation.) (3) CCC will glue the dural hole at the time of the catheter removal, as described below During anesthesia (see FIG. 4A) the epidural needle 14 inserted between spinal ligaments 18 accidentally (or intentionally) can puncture the dural wall 22 into the intrathecal space 26. In this case the catheter with collagen-coated distal end 52 is threaded through the epidural needle (FIG. 4A). When the inserted catheter reached the mark 56, the epidural needle is withdrawn slowly.

The collagen-coated catheter prevents the leakage of the CSF by: (1) The mechanical obstruction of the inner dural hole (glue dural hole from inside); (2) The promotion of cell growth and accelerated closure of the dural hole. [This is how a collagen patch works in spinal surgery; namely, a matrix for the cell growth.]

The collagen covering the distal end 52 of the catheter 50 can have the same properties as Duragen (FIG. 4A, and see the description for collagen dural patch). After the introducing of the CCC in the intrathecal space (FIG. 4B), the collagen 54 on the distal end of the catheter soaks CSF and enlarges (FIG. 4C). It will not quickly dissolve in the CSF, nor is it going to slide off the catheter. [Eventually it will be dissolved, generally in a few days to a few weeks; this time to be determined by in vivo experiments.] The CCC creates a physical barrier for the CSF leak and in this way maintains intrathecal pressure, preventing development of PDPH. Then, when the time comes (a few hours to a few days from the time of the insertion, based on the clinical plan), the catheter can be gently withdrawn (FIG. 4C). The collagen coating slides off the catheter and is “harvested” on the walls of the dural hole, creating the collagen glue. At the same time, the size of the dural hole at the time of the catheter removal is smaller due to the fact that the catheter itself and the collagen over the catheter, that is at the dural hole, promotes cell growth and closure of the hole. The “collagen glue” at the dural hole will undergo degradation and the normal collagen tissue (that constitutes the dural wall) will ultimately substitute for the “collagen glue”, creating the permanent dural patch.

The health care provider may prefer CCC to a regular catheter because: (1) It prevents the development of the PDPH. The placement of the regular epidural catheter in the intrathecal space can also prevent the development of PDPH to some extent, but the risk is still significant. (2) It is an operator friendly technique; no special instructions are required.

As used herein collagen-type material means collagen, fibrin, Duragen, or the like, as well as any natural or synthetic material, presently existing or developed in the future, that is or becomes a substitute or supplement for collagen. In accordance with a feature of the invention, the collagen-type material can contain medication, such as a time-release medication. The medicine can be, for example, a pain reliever, a medicine used to treat infection, a chemotherapy agent, or any other desired medicine.

As demonstrated herein, a device and technique that can be used immediately after dural puncture to prevent the development of PDPH is an important advance in the art.

1. A device for repairing a dural puncture, comprising: a patch formed of collagen type of material that includes a bulbous head portion that is larger than the puncture opening, and a neck portion that is proportioned to fit through the puncture opening.

2. The device as defined by claim 1, wherein said collagen type of material comprises collagen.

3. The device as defined by claim 1, wherein said collagen type of material comprises fibrin.

4. The device as defined by claim 1, wherein said collagen type of material comprises Duragen.

5. The device as defined by claim 1, wherein said patch contains medication.

6. The device as defined by claim 5, wherein said medication is time release medication.

7. The device as defined by claim 1, further comprising a suture tail coupled with said neck portion.

8. A system for sealing a dural puncture, comprising:
a hollow needle;
an elongated catheter within said needle, said catheter having a distal end within the front tip of said needle and a proximal end extending from the rear of said needle; and
a collagen-type material at the distal end of said catheter;
whereby when said needle is inserted through a puncture in the dura mater, the proximal end of the catheter can be manipulated to urge the collagen-type material out of the needle tip to form a seal of the puncture.

9. The system as defined by claim 8, wherein said hollow needle comprises an epidural needle or a spinal needle.

10. The system as defined by claim 8, wherein said collagen-type material comprises a coating of collagen-type material on the distal end of said catheter.

11. The system as defined by claim 8, wherein said collagen-type material comprises a patch formed of collagen type of material that includes a bulbous head portion and a neck portion that is proportioned to fit through the puncture opening.

12. The system as defined by claim 11, wherein said patch is folded to fit within said needle.

13. The system as defined by claim 12, wherein the neck portion of said patch is within the distal end of said catheter.

14. A method for sealing a dural puncture, comprising the steps of:
providing a needle and an elongated catheter within said needle, said catheter having a distal end within the front tip of said needle and a proximal end extending from the rear of said needle;
providing a collagen-type material at the distal end of said catheter;
inserting said needle through a puncture in the dura mater; and
manipulating the proximal end of the catheter to urge the collagen-type material out of the needle tip to form a seal of the puncture.

15. The method as defined by claim 14, wherein said step of providing a needle comprises providing an epidural needle or spinal needle.

16. The method as defined by claim 14, wherein said step of providing a collagen-type material at the distal end of said catheter comprises coating said distal end of said catheter with a collagen-type material.

17. The method as defined by claim 16, wherein said coating of collagen-type material is adapted to be larger than said puncture when it becomes soaked with spinal fluid.

18. The method as defined by claim 14, wherein said step of providing a collagen-type material at the distal end of said catheter comprises providing a patch formed of collagen type of material that includes a bulbous head portion and a neck portion that is proportioned to fit through the puncture opening.

19. The method as defined by claim 18, wherein said patch is folded to fit within said needle.

20. The method as defined by claim 14, wherein said step of providing a collagen-type material comprises providing a material selected from the group consisting of collagen, fibrin, and Duragen.
